(19) United States
(12) Patent Application Publication
(10) Pub. No.: US 2020/0085345 A1
(43) Pub. Date: Mar. 19, 2020

(54) OBJECT INFORMATION ACQUISITION APPARATUS AND METHOD OF CONTROLLING THE SAME

(71) Applicant: CANON KABUSHIKI KAISHA, Tokyo (JP)

(72) Inventors: Ryuichi Nanaumi, Tokyo (JP); Kazuhiko Fukutani, Yokohama-shi (JP)

(21) Appl. No.: 16/563,250

(22) Filed: Sep. 6, 2019

(30) Foreign Application Priority Data
Sep. 13, 2018 (JP) 2018-171759

(51) Int. Cl. A61B 5/103; G01N 29/24; A61B 5/00

(52) U.S. Cl. CPC A61B 5/103; A61B 5/742; A61B 5/0095; G01N 29/2418

(57) ABSTRACT

An object information acquisition apparatus includes: a probe receiving an acoustic wave generated from an object and to output a signal; a scanning unit scanning the object with the probe by changing a position of the probe relative to the object; a control unit controlling execution of acoustic wave measurement on the object; and an information acquiring unit acquiring characteristic information on the object by using the signal, wherein the control unit executes first acoustic wave measurement and second acoustic wave measurement for a predetermined region of the object, and the first acoustic wave measurement is executed within a shorter period of time than the second acoustic wave measurement, and the information acquiring unit acquires displacement information indicating displacement of the object by using a first signal acquired by the first acoustic wave measurement and a second signal acquired by the second acoustic wave measurement.

### BACKGROUND OF THE INVENTION

### Field of the Invention

The present invention relates to an object information acquisition apparatus and a method of controlling the same.

### Description of the Related Art

As technology for obtaining information on an object such as a living body by receiving acoustic waves, object information acquisition apparatus such as photoacoustic imaging apparatus and ultrasonic echo imaging apparatus has been proposed.

Photoacoustic imaging apparatus, in particular, has been shown to be useful in the diagnosis of skin diseases and breast cancer. When irradiating object's tissue with visible light or near-infrared light, the light absorbing materials inside the object (e.g., hemoglobin in the blood, etc.) are instantaneously expanded by absorbing light energy and generate acoustic waves. This phenomenon is called photoacoustic effect, and the generated acoustic wave is also known as photoacoustic wave. Photoacoustic imaging apparatus visualizes information of the object's tissue by measuring the photoacoustic waves.

This tomographic technology using photoacoustic effects is called photoacoustic imaging (PAI). Photoacoustic imaging can visualize information on the absorption coefficient inside the object. The absorption coefficient means a rate indicating how much light energy is absorbed by an object's tissue. Information related to the absorption coefficient includes, for example, initial sound pressure which is sound pressure at the moment the photoacoustic wave occurs. The initial sound pressure is proportional to the product of the light energy (light intensity) and the absorption coefficient. Furthermore, the absorption coefficient depends on the concentration of the components in the object's tissue. Therefore, the concentration of these components can be obtained from the absorption coefficient. Information such as oxygen saturation can also be obtained from the concentration information. By analyzing these information, application to medical diagnosis is expected such as distinguishing tumor tissue and the surrounding tissue in the object.

Furthermore, ultrasonic echo imaging apparatus is used in various fields including diagnosis since morphological information can be obtained by visualizing a difference in acoustic impedance inside the object.

In object information acquisition apparatus using probes or transducers for scanning, images are reconstructed with signals obtained at each scanned points (measurement position) on the scanning path. One of the challenges in scanning-type object information acquisition apparatus is a displacement of the object during scanning. That is, when a displacement occurs due to body movements of the object during scanning, it is possible that the accuracy of the object information decreases.

In Japanese Patent Application Publication No. 2014-061124, a photoacoustic measuring apparatus for determining the positional deviation of the ultrasonic probe is disclosed. Further, “Motion correction in optoacoustic mesoscopy”, Scientific Reports 7, article number: 10386 (2017) (hereinafter referred to as Non-Patent Document 1) discloses a method of correcting the body movement of the object in the photoacoustic imaging apparatus.

### SUMMARY OF THE INVENTION

Photoacoustic measuring apparatus described in Japanese Patent Application Publication No. 2014-061124 is equipped with a member having a pattern formed thereon in order to determine the positional deviation of the probe. However, when the member is used for the detection of body movement, there is a possibility that the image quality is reduced because artifacts are introduced by the pattern on the member or sound velocity of the member is different from that of the object. In addition, by using the method described in Non-Patent Document 1, while body movements in sufficiently short period relative to the total scanning time can be corrected, it is difficult to correct long-period body movements occurring over the total scanning time.

The present invention has been made in view of the above problems. An object of the present invention is to reduce the influence of displacement of the object in apparatus for receiving acoustic waves.

The present invention adopts the following configuration, that is: an object information acquisition apparatus includes: a probe configured to receive an acoustic wave generated from an object and to output a signal; a scanning unit configured to scan the object with the probe by changing a position of the probe relative to the object; a control unit configured to control execution of acoustic wave measurement on the object; and an information acquiring unit configured to acquire characteristic information on the object by using the signal, wherein the control unit executes first acoustic wave measurement and second acoustic wave measurement for a predetermined region of the object, and the first acoustic wave measurement is executed within a shorter period of time than the second acoustic wave measurement, and the information acquiring unit acquires displacement information indicating displacement of the object by using a first signal acquired by the first acoustic wave measurement and a second signal acquired by the second acoustic wave measurement.

The present invention also adopts the following configuration, that is: a method of controlling an object information acquisition apparatus including a probe, a scanning step, a control step and an information acquisition step, includes: an output step of receiving, by the probe, an acoustic wave generated from an object and outputting a signal; the scanning step of scanning the object with the probe by changing a position of the probe relative to the object; the control step of controlling execution of acoustic wave measurement on the object; and the information acquisition step of acquiring characteristic information on the object by using the signal, wherein the control step includes executing first acoustic wave measurement and second acoustic wave measurement for a predetermined region of the object, and the first acoustic wave measurement is executed within a shorter period of time than the second acoustic wave measurement, and the information acquisition step includes acquiring displacement information indicating displacement of the object by using a first signal acquired by the first acoustic wave measurement and a second signal acquired by the second acoustic wave measurement.

According to the present invention, the influence of the displacement of the object can be reduced in apparatus for receiving acoustic waves.

Further features of the present invention will become apparent from the following description of exemplary embodiments with reference to the attached drawings.

### BRIEF DESCRIPTION OF THE DRAWINGS

FIGS. 1A and 1B are a schematic diagram of an object information acquisition apparatus of a first embodiment and a schematic diagram around its processing unit, respectively;

FIG. 2A is a flowchart illustrating the overall process of the first embodiment;

FIG. 2B is a flowchart of an object information acquisition method of the first embodiment;

FIGS. 3A and 3B are typical examples of first and second acoustic wave measurements of the first embodiment;

FIGS. 4A to 4D are schematic diagrams of a calculating method of body movement correction amount of the first embodiment;

FIGS. 5A to 5C are schematic diagrams illustrating the body movement correction method;

FIGS. 6A to 6E are diagrams illustrating examples of the display unit;

FIGS. 7A to 7D are diagrams illustrating a case where the body movement amount exceeds an acceptable threshold;

FIGS. 8A to 8H are diagrams illustrating scanning paths of the first acoustic wave measurement;

FIG. 9 is a flowchart of an object information acquisition method of the second embodiment;

FIGS. 10A and 10B are diagrams of typical examples of first and second acoustic wave measurements of the second embodiment;

FIGS. 11A to 11E are schematic diagrams illustrating a calculating method of body movement correction amount of the second embodiment;

FIGS. 12A and 12B are a schematic diagram of the object information acquisition apparatus of the third embodiment and a schematic diagram around its processing unit, respectively;

FIG. 13 is a flowchart of an object information acquisition method of the third embodiment;

FIGS. 14A and 14B are diagrams illustrating typical examples of first and second acoustic wave measurements of the third embodiment;

FIGS. 15A to 15C are schematic diagrams of an object in cross section and amounts of the light reached;

FIG. 16 is a schematic diagram illustrating the relation between pulse frequency and pulse energy of the light source; and

FIG. 17 is a schematic diagram of another configuration example of the object information acquisition apparatus.

### DESCRIPTION OF THE EMBODIMENTS

Referring to the drawings, preferred embodiments of the present invention are described below. Note that the dimensions, materials, and shapes of the components described below and relative arrangements thereof are intended to be appropriately changed depending on configurations and various conditions of an apparatus to which the present invention is applied. Therefore, the scope of the present invention is not intended to be limited to the following description.

The present invention relates to a technology irradiating an object with light (electromagnetic waves) to generate and obtain characteristic information inside the object (object information) using acoustic waves emitted from inside of the object. Therefore, the present invention is perceived as a photoacoustic apparatus or a control method thereof, an object information acquisition apparatus or a control method thereof, or an object information acquisition method or a signal processing method. The present invention also includes a program for executing these methods in an information-processing device comprising hardware resources such as a CPU or a memory as well as a computer-readable non-transitory storage medium that stores the program.

Furthermore, the present object information acquisition apparatus includes devices using echo technology which receives (detects) acoustic waves being emitted toward an object, reflected and scattered on a specific position in the object, and propagated. Since such an object information acquisition apparatus obtains characteristic information inside the object based on reflective and scattering properties of the acoustic wave in the form of image data, it can also be referred to as ultrasonic echo imaging apparatus. In this case, the present invention is perceived as an ultrasonic echo imaging apparatus or a control method thereof, an object information acquisition apparatus or a control method thereof, or an object information acquisition method or a signal processing method. The present invention is also perceived as a program for executing these methods in an information-processing device comprising hardware resources such as a CPU or a memory as well as a computer-readable non-transitory storage medium storing the program.

The characteristic information of the photoacoustic device is a value reflecting the absorption amount or absorption rate of the optical energy, which is generated by received signals derived from photoacoustic waves and corresponds to each of a plurality of positions inside the object. The characteristic information includes, for example, a source of the acoustic waves caused by the light irradiation of a single wavelength, initial sound pressure in the object, and absorption density or absorption coefficient derived from the initial sound pressure. Furthermore, it is possible to obtain concentration of the materials constituting the tissue from the characteristic information obtained by a plurality of wavelengths different from each other. By calculating the oxidized hemoglobin concentration and reduced hemoglobin concentration in the tissue, it is possible to derive the oxygen saturation distribution. The concentration of the materials such as glucose concentration, collagen concentration, and melanin concentration as well as the volume fraction of fat or water can also be determined.

Furthermore, the characteristic information in the ultrasonic echo imaging apparatus refers to information indicating acoustic impedance differences in the object as well as positional information with the acoustic impedance difference and its sound velocity and density etc.

Based on the characteristic information at each position in the object, two-dimensional or three-dimensional distribution of the characteristic information is obtained. Distribution data can be generated as image data. Characteristic information can be derived not only as numerical data but also as distribution information at each position in the object. That is, distribution information such as the distribution of the initial sound pressure, energy absorption density, absorption coefficient or oxygen saturation. Alternatively, in the case of ultrasonic echo imaging, it is distribution information on the acoustic impedance.

The acoustic wave referred to in the present invention typically indicates an ultrasonic wave and includes an elastic wave called a sound wave or acoustic wave. Electrical signals converted from the acoustic wave by a transducer and the like is also referred to as acoustic signals. However, the wording ultrasonic or acoustic waves herein is not intended to limit the wavelength of their elastic waves. Acoustic waves generated by photoacoustic effect are called photoacoustic waves or photoultrasonic waves. Electrical signals derived from photoacoustic waves are also referred to as photoacoustic signals. Acoustic waves generated in ultrasonic echo imaging are also referred to as ultrasonic echoes or echo waves. Electrical signals derived from ultrasonic echoes are also referred to as ultrasonic signals and echo signals. Distribution data is also referred to as photoacoustic image data, ultrasonic image data or reconstructed image data.

The object information acquisition apparatus of the present invention is suitable for diagnosing vascular diseases and malignant tumors as well as following up chemotherapies in humans and animals. Examples of the object are a part of a living body such as a breast or hand of an object, non-human animals such as mice, inanimate, phantoms, and the like.

### First Embodiment

Referring to the drawings, the embodiments of the present invention are described in detail below. The same components are denoted by the same reference symbols in principle, and descriptions thereof are omitted.

Configuration of Object Information Acquisition Apparatus

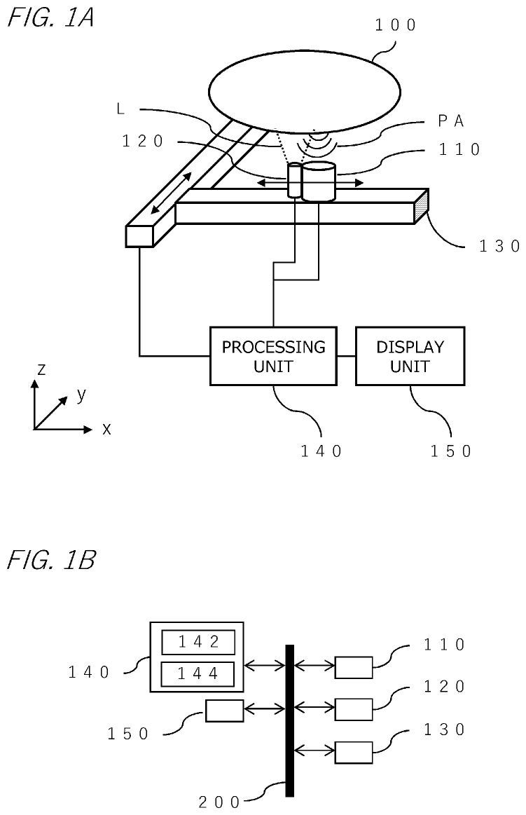

FIG. 1A is a schematic diagram of the object information acquisition apparatus according to the present embodiment. Each component of the apparatus is now described. The apparatus includes a probe 110, an irradiation unit 120, a scanning unit 130, a processing unit 140, and a display unit 150. A measurement target is an object 100. FIG. 1B is a schematic diagram illustrating the relation between the processing unit 140 and the peripheral configuration.

The processing unit 140 controls the operation of each component of the object information acquisition apparatus via a bus 200. The processing unit 140 stores therein a program in which an object information acquisition method described below is written, and reads the program to cause the object information acquisition apparatus to execute the object information acquisition method.

The irradiation unit 120 irradiates the object 100 with light L. Then, photoacoustic waves PA are generated from the inside of the object and from the surface of the object by photoacoustic effect. The probe 110 receives the propagated acoustic waves to obtain electrical signals in time series as received signals. The processing unit 140 performs processing on the received signals and generates image data to be displayed on the display unit 150.

Detailed Descriptions of Components

Details of each configuration in the object information acquisition apparatus according to the present embodiment are described below.

Object 100

An object, although it does not constitute a part of the object information acquisition apparatus of the present invention, is described below. The main objective of the object information acquisition apparatus of the present invention is to diagnose diseases such as vascular diseases and malignant tumors as well as to follow up chemotherapies in humans and animals. Therefore, the object is expected to be diagnostic target areas of a living body, specifically such as limbs, fingers, breasts, head, neck, and abdomen of humans and animals.

The light absorbing materials inside the object are assumed to be relatively high in the optical absorption coefficient. For example, if a human body is to be measured, the light absorbing materials to be targeted for the measurement are oxyhemoglobin, deoxyhemoglobin, blood vessels that contain high level of oxyhemoglobin or deoxyhemoglobin, malignant tumors including many neovasculars, and normal and abnormal skin containing pigments such as melanin other than blood vessels. In addition, plaques on the carotid arterial wall can also be measured.

Probe 110

The probe 110 includes a transducer, which is an element capable of detecting acoustic waves. A transducer can receive acoustic waves and convert the acoustic waves into electrical signals, which are analog signals. The probe or transducer is also referred to as acoustic probe, probe, acoustic wave probe, acoustic wave sensing element, acoustic wave detector, acoustic wave receiver, or the like. The probe 110 can be anything if it can receive acoustic waves, such as those using piezoelectric phenomena, the resonance of light, or changes in capacitance. Acoustic waves used in the present embodiment typically consist of frequency components from several hundred KHz to 100 MHz. Therefore, as the transducer, it is preferable to use those capable of detecting these frequencies. Likewise, it is desirable to use a probe with high sensitivity and a wide frequency band. The examples of a probe include those with piezoelectric elements using lead zirconate titanate (PZT), polymer piezoelectric film materials such as polyvinylidene fluoride (PVDF), capacitive micromachined ultrasonic transducer (CMUT), and a Fabry-Perot interferometer.

The number of transducers provided on the probe 110 can be a single or plural. For a single transducer, the shape can be rectangular, circular, planar, spherical, or elliptical. Photoacoustic microscopes using a single element transducer are also object to application of the present invention.

For a plurality of transducers may be used in such way as an array transducer in which a plurality of transducers is arranged in 1D, 1.5D, or 2D. Alternatively, a plurality of transducers may be arranged on a bowl-like or spherical cap-like support in order to form a high sensitivity area where pointing axes of each transducer converge. Also, the support of the array transducer or the bowl-like support can be integrated with the irradiation unit for irradiating an object with light so as to be simultaneously movable.

Irradiation Unit 120

The irradiation unit 120 irradiates the object 100 with light generated from a light source (not shown). The irradiation unit 120 can operate by the control of the processing unit 140 or, in cooperation with the processing unit 140, by the control of the control circuit equipped in the irradiation unit itself. In the former case, the processing unit 140 also serves as an irradiation control unit; in the latter case, the irradiation unit is equipped with an irradiation control unit. Alternatively, an irradiation control unit can be separately provided. The irradiation control unit controls the irradiation conditions such as timing and a light amount of the irradiation, a pulse length and interval of the pulsed light, and a wavelength of the irradiation light by obtaining the irradiation control information specified by a user or stored in advance in a memory. Further, the irradiation control unit can control an irradiation position by linking with position control information of the scanning control unit. The irradiation unit corresponds to the irradiation means in the present embodiment.

The irradiation unit 120 typically consists of an optical system comprising optical components such as lenses and mirrors. The irradiation unit 120 irradiates the object 100 with light by shaping the light into a desired distribution shape. The following or the like can be used as the optical component: a waveguide such as an optical fiber for propagating light; a mirror for reflecting light; a lens for attracting, enlarging light, or for changing the shape of light; a prism for dispersing, refracting, or reflecting light; and a diffusion plate for diffusing light. Any optical component can be used as long as the object can be irradiated with the light emitted from the light source and having a desired shape. Note that any light source can be regarded as the irradiation unit 120 if the object 100 can be irradiated with original light emitted from the light source as the desired light f.

For the light source (not shown), a pulse light source that can generate pulse light of several nano- to micro-second order is preferred. In order to efficiently generate photoacoustic waves, it is necessary to irradiate the object with light in a sufficiently short time according to the thermal properties of the object. Specifically, it is preferable that the light source is capable of generating light of several hundred nanoseconds or less in pulse width. If the object is a living body, a preferred pulse width of the pulsed light generated from the light source is about 10 to 50 nanoseconds.

It is preferable that the pulsed light is at a specific wavelength which is absorbed by a particular component of the components constituting the object and at which the light propagates inside the object. If the object is a living body, a preferred wavelength is at least 500 nm and not more than 1200 nm, more preferably at least 700 nm and not more than 1100 nm. However, when determining an optical characteristic value distribution of biological tissue relatively in the vicinity of the biological surface, a wider range of wavelengths than the above wavelength range (e.g., at least 400 nm and not more than 1600 nm) can be used. For a light source, lasers, flash lamps, and light emitting diodes can be used. Various lasers such as solid-state lasers, gas lasers, dye lasers, and semiconductor lasers can be used as the laser. For example, Alexandrite laser, Yttrium-Aluminium-Garnet laser, Titanium-Sapphire laser may be used.

Here, while the light sources for generating light have been mentioned, it may also be means for generating electromagnetic waves. For example, the object information can also be obtained with a microwave source in the same principle as photoacoustic imaging.

Scanning Unit 130

The scanning unit 130 (scanning unit) scans the object 100 with the probe 110 and changes its relative position to the object 100. Similarly, the scanning unit 130 scans the object 100 with the irradiation unit 120 and changes its relative position to the object 100. The scanning can be performed by linking the probe 110 and the irradiation unit 120 or independently by separating the probe 110 and the irradiation unit 120. When linking the movement of the probe 110 and the irradiation unit 120, they may be combined and moved integrally. The scanning unit corresponds to the scanning unit in the present embodiment.

The scanning unit 130 in FIG. 1A scans the xy plane with the probe 110 and the irradiation unit 120 which are combined integrally. The scanning unit 130 can operate by the control of the processing unit 140 or, in cooperation with the processing unit 140, by the control of the control circuit equipped in the irradiation unit itself. In the former case, the processing unit 140 also serves as a scanning control unit; in the latter case, the scanning unit equips a scanning control unit. Alternatively, a scanning control unit can be separately provided. The scanning control unit controls the scanning conditions such as the scanning paths, the timing of start and end of the scan, and the speed at scanning by obtaining the scanning control information specified by a user or stored in advance in a memory. Further, the scanning control unit can control the position and timing of the irradiation by linking with irradiation control information of the irradiation control unit.

If the processing unit 140 controls scanning, the processing unit 140 (control unit) stores positions (measurement positions) which are irradiated with electromagnetic waves and at which an acoustic waves are received in a memory as coordinate values by using a position information acquiring unit such as an encoder. Measurement position information is used in the imaging process of the object. While the present embodiment describes scanning an xy plane, a three-dimensional scanning including the z direction may be performed instead.

As a scanning path, various paths such as raster trajectory, spiral trajectory, or circular trajectory can be used. Acoustic wave measurement (i.e., light irradiation from the irradiation unit 120 and acoustic wave reception by the probe 110) is carried out at the measurement position on these paths. The term “measurement position” or “scanning point” in the acoustic wave measurement refers to a position on a scanning path which is irradiated with light and at which an acoustic wave generates. Here, if using a step-and-repeat method where the probe repeats the process of pause, acoustic wave measurement, and relocation, the position at which the probe is paused is defined as the measurement position. Further, if using a method where the probe moves continuously, the measurement position may be a position of the probe when light is applied, or any position in the period for receiving photoacoustic waves generated by the light irradiation (e.g., position of the probe at the midpoint during the reception period).

Alternatively, the handheld probe 110 can also be handled manually for the scanning. In this case, it is preferable to obtain the spatial position or posture of the probe as positional information on the probe. The spatial position and posture of the probe can be obtained, for example, by a motion capture camera, position capture device by magnetism.

Processing Unit 140

The processing unit 140 (information acquiring unit) performs computation using received signals in order to obtain object information inside the object. Typically, it consists of elements such as CPUs and GPUs and circuits such as FPGAs and ASICs. It is preferable that the processing unit 140 equips a memory that stores a program, control information, results of acoustic wave measurement, or the like. It should be noted that the processing unit 140 may be not only composed of one element or circuit but also of a plurality of elements and circuits. Further, any element or circuit may perform any process(es) described in the object information acquisition method. An apparatus that executes each process is collectively referred to as the processing unit according to the present embodiment. For the processing unit 140, workstations and personal computers can typically be used. When using a workstation or the like as the processing unit 140, the UI of the workstation (e.g., keyboard, mouse, touch panel, etc.) may accept the input of the instruction information from a user. The processing unit corresponds to the control unit and information acquiring unit in the present embodiment. It should be noted that, as shown in FIG. 1B, a control unit 142 and an information acquiring unit 144 may be implemented as a functional block constituting the processing unit 140.

The processing unit 140 may include an A/D converter or signal amplifier. The A/D converter converts analog electrical signals converted from acoustic waves by the probe 110 into digital signals. The signal amplifier processes the received signals to amplify. Further, the A/D converter and the signal amplifier may be provided as a separate signal processing unit from the processing unit 140.

It is preferable that the processing unit 140 is configured to be able to simultaneously perform pipeline processing on a plurality of signals. This makes it possible to shorten the time to obtain the object information. Further, the processing unit 140 has a non-transitory recording medium, which can store each process performed by the object information acquisition method as a program to be executed by itself.

The processing unit 140 may be provided in a configuration contained in the same enclosure with the probe 110. However, the processing unit 140 may perform some part of the signal processing in a processing unit contained in the enclosure, and perform the remaining signal processing in a processing unit provided outside the enclosure. In this case, processing units provided internally and externally of the enclosure are collectively referred to as the processing unit according to the present embodiment. In addition, the arrangement of each component of the object information acquisition apparatus shown is an example, and it may be any arrangement as long as it can perform the processing required for the present invention as a whole.

Display Unit 150

The display unit 150 is a device for displaying the object information, which is characteristic information output from the processing unit 140. For the display unit 150, for example, a liquid crystal display, a plasma display, an organic EL display, or FED can be used. It should be noted that the display unit 150 or the processing unit 140 may perform image processing such as adjusting the luminance value when displaying the object information. Further, in addition to the object information, the processing unit 140 may display instructions and messages to the operator or the object on the display unit 150. The display unit corresponds to the display means in the present embodiment.

Object Information Acquisition Method

Next, with reference to the drawings, each step of the object information acquisition method according to the present embodiment will be described. Note that each step is executed by controlling the operation of each configuration of the object information acquisition apparatus by the processing unit 140.

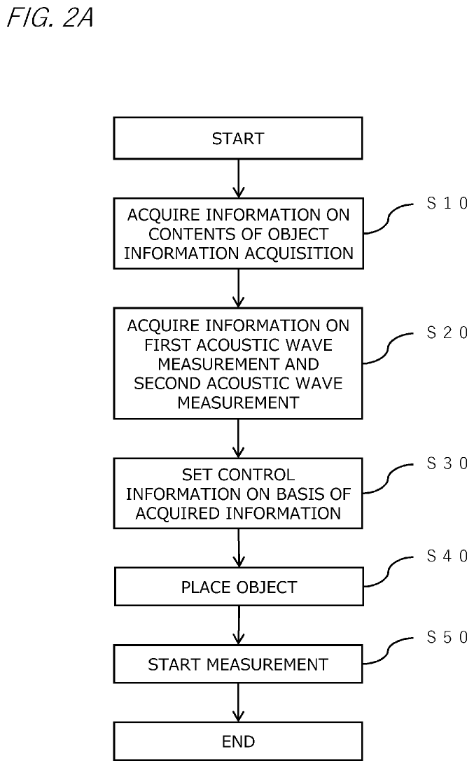

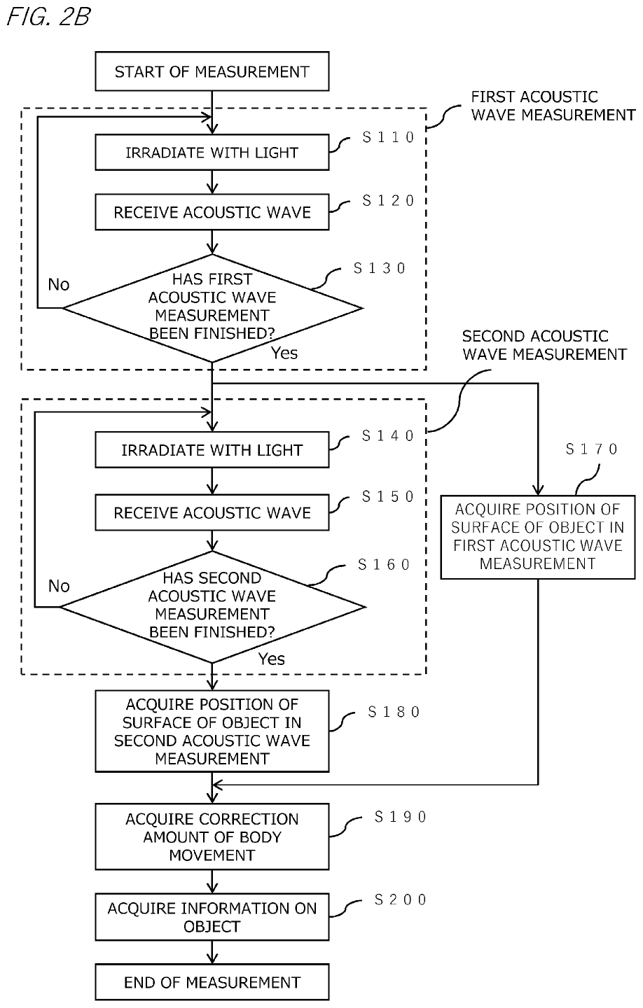

FIG. 2A is a flowchart illustrating the overall process.

Step S10

The processing unit 140 acquires information on the contents of the object information acquisition. This information includes, for example, a type of the object, size and depth of a predetermined area of interest in the object, a type of the object information, a desired accuracy of the object information, and various information on the acoustic wave measurement. The processing unit 140 acquires information on the contents of the acoustic wave measurement by obtaining information input by a user or reading information stored beforehand in a memory.

Step S20

The processing unit 140 acquires information on a first and a second acoustic wave measurement described below. The information on the acoustic wave measurement contains at least a scanning path, a position of the acoustic wave measurement on the path. The position of acoustic wave measurement includes a position irradiated with light and a position for acoustic wave reception. Information on the acoustic wave measurement can also be obtained in any manner such as information input by a user or stored in a memory. It should be noted that the steps S10 and S20 may be combined as one step.

Step S30

The processing unit 140 sets control information of the apparatus based on the information on the contents of the object information acquisition and the acoustic wave measurement. The control information includes at least irradiation control information and scanning control information. The processing unit 140 calculates a moving direction and distance by the scanning unit 130, a timing of the light irradiation and a timing of acoustic wave reception at each timing after the measurement starts based on the path and measurement position in the first and second acoustic wave measurement. Based on the information derived above, the processing unit 140 calculates and sets parameters such as the scanning control information on the scanning unit 130 and the irradiation control information on the irradiation unit 120. Further, the processing unit 140 calculates and sets parameters of the reception control information on the reception of the acoustic wave by the transducer of the probe. Note that the processing unit 140 may set the parameters by reading pre-stored control information. In that case, the parameters used in the measurement may be selected from a plurality of default parameters depending on types of the object or specification of a user.

Step S40

An operator places the object on a predetermined position.

Step S50

An operator checks the setting of the control information and completion of the placement of the object and starts the acoustic wave measurement described in FIG. 2B.

FIG. 2B is a flowchart illustrating the object information acquisition method in the present embodiment. Steps S110 to S130 constitute the first acoustic wave measurement and steps S140 to S160 constitute the second acoustic wave measurement. In the first acoustic wave measurement, when considering the movement of the probe, the name first scan is used. Similarly, in the second acoustic wave measurement, when considering the movement of the probe, the name second scan is used. The acoustic wave and acoustic signal obtained in the first acoustic wave measurement can be referred to as the first signal. Similarly, the acoustic wave and acoustic signal obtained in the second acoustic wave measurement can be referred to as the second signal.

Step S110: Step for Generating Photoacoustic Wave by Irradiating Inside of Object with Light

The irradiation unit 120 irradiates the object 100 with light. Then, the light absorbing materials inside and on the surface of the object absorb light energy and generate a photoacoustic wave.

Step S120: Step for Obtaining Received Signal by Receiving Photoacoustic Wave

The transducer of the probe 110 receives (detects) the photoacoustic wave, and outputs the received signal to the processing unit 140.

Step S130: Step for Determining Completion of First Acoustic Wave Measurement

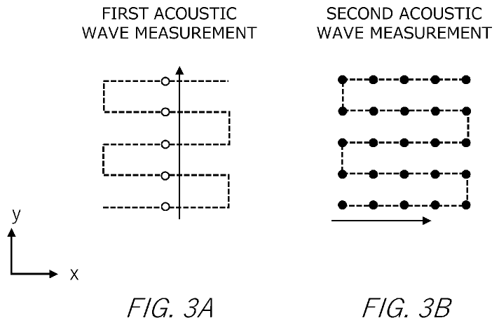

In this step, the processing unit 140 determines whether to complete the first acoustic wave measurement. Specifically, the processing unit 140 repeats the steps S110 and S120 by scanning an object with the probe 110 and/or the irradiation unit 120 until the measurements are completed at all the measurement positions (white circles in FIG. 3A) where the received signals should be obtained in the first acoustic wave measurement. If there are no unmeasured measurement positions left, the processing unit 140 terminates the first acoustic wave measurement and proceeds to the second acoustic wave measurement.

Step S140: Step for Generating Photoacoustic Wave by Irradiating Inside of Object with Light

Step S150: Step for Obtaining Received Signal by Receiving Photoacoustic Wave

In the second acoustic wave measurement, these processes are carried out in the same manner as the processing of the steps S110 and S120.

Step S160: Step for Determining Completion of Second Acoustic Wave Measurement

In this step, the processing unit 140 determines whether to complete the second acoustic wave measurement. Specifically, the processing unit 140 repeats the steps S140 and S150 by scanning an object with the probe 110 and/or the irradiation unit 120 until the measurements are completed at all the measurement positions (white circles in FIG. 3B) where the received signals should be obtained in the second acoustic wave measurement. If there are no unmeasured measurement positions left, the processing unit 140 terminates the second acoustic wave measurement.

Here, with reference to FIGS. 3A and 3B, the first and the second acoustic wave measurements will be described. The dashed lines in FIGS. 3A and 3B indicate scanning paths in a raster trajectory. FIG. 3B represents the state of the second acoustic wave measurement and the black circles show each measurement position. The processing unit 140 generates object information using the received signals obtained at each measurement position of the second acoustic wave measurement.

FIG. 3A represents the state of the first acoustic wave measurement and the white circles show each measurement position. The number of measurement positions in the first acoustic wave measurement is fewer than the number of measurement positions in the second acoustic wave measurement. Therefore, the first acoustic wave measurement is completed in a shorter period of time than the second acoustic wave measurement. Thus, it is possible to obtain received signals with less influenced by the long-period body movements in the first acoustic wave measurement. In the example of FIGS. 3A and 3B, the first scan is performed by following a linear path of which direction is orthogonal to the main scanning direction of the second scan.

The long-period body movement, unlike the sudden short-term body movement of the object, is referred to a gradual change of the posture because, for example, it is difficult for an object to maintain the posture for a long time. For example, if the test site is a foot of the object and it is necessary to take a posture with the foot lifted for the measurement, the position of the foot would be gradually displaced (lowered) due to fatigue when the measurement continues over a long period of time. However, the distinction between the long-period and the short-period body movement is relative and is not to be determined by time or measurement content. Of the entire time required for acoustic wave measurement, if the period of the body movement is long enough to affect the accuracy of the object information, it is an object to correction according to the present invention.

Step S170: Step for Acquiring Surface Shape Information in First Acoustic Wave Measurement

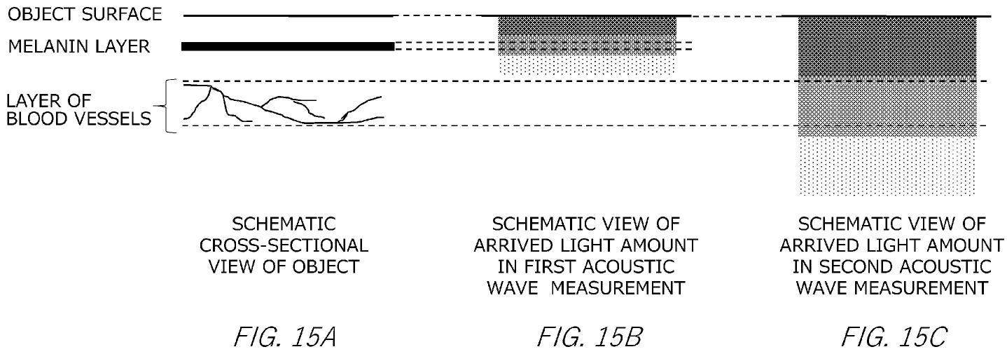

In this step, by using the received signals obtained in steps S110 to S130, the object surface position (z coordinate) is obtained and defined as first surface shape information. The surface shape information is information indicating a displacement on the z direction of the object at a certain timing. In the present embodiment, the total processing time is shortened since the processing of S170 is performed in parallel with the second acoustic wave measurement. However, the processing of S170 can be performed at any time such as before or after the second acoustic wave measurement as long as it is after the completion of the first acoustic wave measurement. Here, the term object surface is not necessarily intended to mean exactly the outermost surface of the object. Any site can be used as a reference site as long as it can obtain effective object information in order to detect a displacement of the object. The information of such reference sites may be used as a target for the information acquisition in S170 and S180. For example, as shown in FIGS. 15A to 15C below, there is a melanin layer deeper than the outermost surface of the object. By obtaining the object information of such melanin layer, it may be used as a basis for calculating the body movement amount. The object surface position to be obtained in S170 or S180 may be referred to as a reference site position. Surface shape information in the first acoustic wave measurement can be referred to as the first object shape information.

To obtain the object surface position in S170, for example, the method described in Non-Patent Document 1 can be used. That is, the processing unit 140 detects signal components derived from a melanin layer near the object surface from the received signal of in time-series. Then, by multiplying the sound speed and a time when the signal from the object surface is detected, a distance from the object surface to the transducer is obtained. By performing this process on the entire object, the surface shape information is obtained. The obtained first surface shape information is information in which the timings of the measurements are different for each measurement position.

The above method allows to obtain the surface shape information without providing a new component to the apparatus. However, the method is not limited thereto as long as the shape information of the object surface is obtained. For example, the surface shape may be obtained by acquiring an optical image from an image-taking device. Further, the surface shape can also be obtained based on a time between an ultrasonic wave is emitted from the transducer toward the object and the echo wave returns to the transducer.

Step S180: Step for Acquiring Surface Shape Information in Second Acoustic Wave Measurement

In this step, by using the received signals obtained in steps S140 to S160, the object surface position (z coordinate) is obtained and defined as second surface shape information. The same method as S170 is used to obtain the object surface position. Surface shape information in the second acoustic wave measurement can be referred to as the second object shape information.

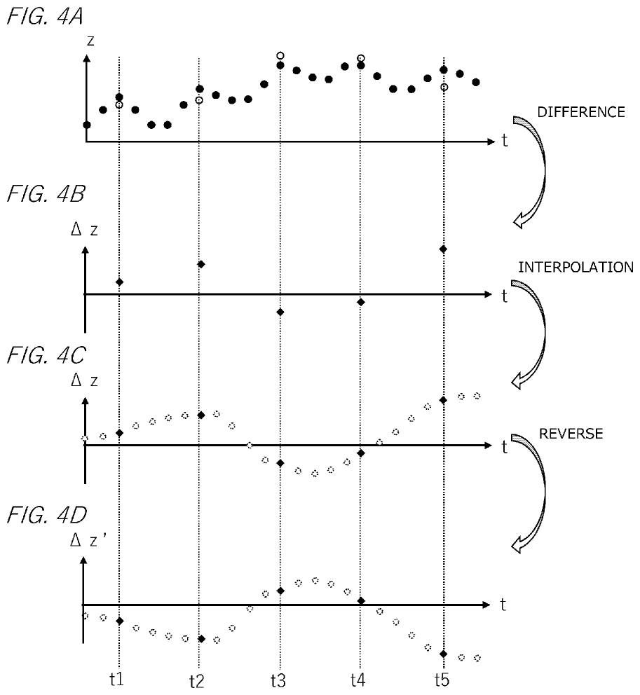

Processing unit 140 combines the first and second surface shape information. Specifically, the processing unit 140 plots the object surface position of each measurement position in the second acoustic wave measurement obtained in S180 with respect to time t, which indicates a time at which the first acoustic wave measurement was performed. Note that since the duration of the first acoustic wave measurement is shorter than those of the second acoustic wave measurement, time correction is performed so as to adjust the measurement timing of the first and the second acoustic wave measurement when plotting relative to time as shown in FIGS. 4A to 4D. When plotting relative to the measurement position or the scanning distance, the measurement positions or the scanning distances of the first and the second acoustic wave measurement are adjusted.

FIG. 4A shows a plot of the object surface positions in the first and second acoustic wave measurement. The vertical axis in FIG. 4A shows the object surface position and the unit is, for example, [mm]. The horizontal axis indicates time, and the unit is, for example, [s]. Note that the time on the horizontal axis is based on the time of the second acoustic wave measurement. The black circles in FIG. 4A represent the object surface positions at each measurement position of the second acoustic wave measurement. White circles in FIG. 4A represent the object surface position at each measurement positions of the first acoustic wave measurement plotted on the time at which the measurement position is the closest to those of the second acoustic wave measurement. Note that when setting the measurement positions in the acoustic wave measurement, it is preferable that each measurement position in the first acoustic wave measurement overlaps either of those in the second acoustic wave measurement.

Step S190: Step for Acquiring Body Movement Correction Amount

In this step, a body movement correction amount is acquired by using the object surface position obtained in S170 and S180. The vertical axis in FIG. 4B represents a body movement amount Δz corresponding to a displacement amount of the object surface in the z direction, and the unit is, for example, [mm]. The body movement amount is considered to be displacement information indicating the amount of displacement of the object.

In S190, the processing unit 140 first extracts a time point where the first acoustic wave measurement corresponding to the measurement position of the second acoustic wave measurement exists. From FIG. 4A, five measurement timings, t1 to t5, are extracted. It should be noted that the same method is used to extract corresponding positions when plotting relative to the measurement position and scanning distance rather than the time.

Subsequently, the processing unit 140 obtains the body movement amount Δz in the z direction by subtracting the object surface position in the first acoustic wave measurement from those in the second acoustic wave measurement. This is shown in FIG. 4B. The body movement amount obtained here can be referred to as displacement information indicating the displacement of the object.

The above process provides the benefit of being able to obtain information on a long-period displacement of the object, which has not been assumed in the past.

Subsequently, the processing unit 140 performs an interpolating process of body movement amount plotted in FIG. 4B with respect to the time of each measurement position in the second acoustic wave measurement. FIG. 4C shows a plot after the interpolating process. As shown in FIG. 4C, it is preferable to determine an interpolation value at all measurement positions in the second acoustic wave measurement.

Subsequently, the processing unit 140 obtains the body movement correction amount Δz′ shown in FIG. 4D by reversing the sign in FIG. 4C.

The above process provides the benefit of being able to obtain the correction information to reduce the influence of the displacement by correcting the long-term displacement of the object.

Step S200: Step for Acquiring Object Information

In this step, the initial sound pressure distribution p₀(r) in the object 100 is calculated using the body movement correction amount obtained in S190.

Here, r represents a position vector of the position where the image is to be reconstructed, and r₀ represents a position vector of the measurement position. dΩ₀ represents a solid angle at which the transducer connects with the position r, and Ω₀ represents the sum of solid angles of the total measurement position.

p(r₀,t̅)

represents the received signals in time-series with respect to time t.

t̅

represents the time converted in distance by multiplying the time t and the sound speed.

At this point, according to “Universal back-projection algorithm for photoacoustic computed tomography”, PHYSICAL REVIEW E 71, 016706 (2005), the initial sound pressure distribution p₀(r) in the object 100 can be derived from the received signals by using the backprojection method in formula (1). This process is referred to as image reconstruction.

$${p}_{0} ( r ) = {\int }_{{Ω}_{0}}^{ } b ( {r}_{0} , \overline{t} = \vert r - {r}_{0} \vert ) d {Ω}_{0} / {Ω}_{0} \\ b ( {r}_{0} , \overline{t} ) = 2 p ( {r}_{0} , \overline{t} ) - 2 \overline{t} \frac{\partial p ( {r}_{0} , \overline{t} )}{\partial \overline{t}} \qquad ( 1 )$$

Principle of Correction

Here in S200, formula (2), where the body movement correction amount Δz′ is added to the formula (1) is used.

p₀(r)=∫_{Ω}_{₀}b(r₀,t̅=|r−r₀|+Δz′)dΩ₀/Ω₀ (2)

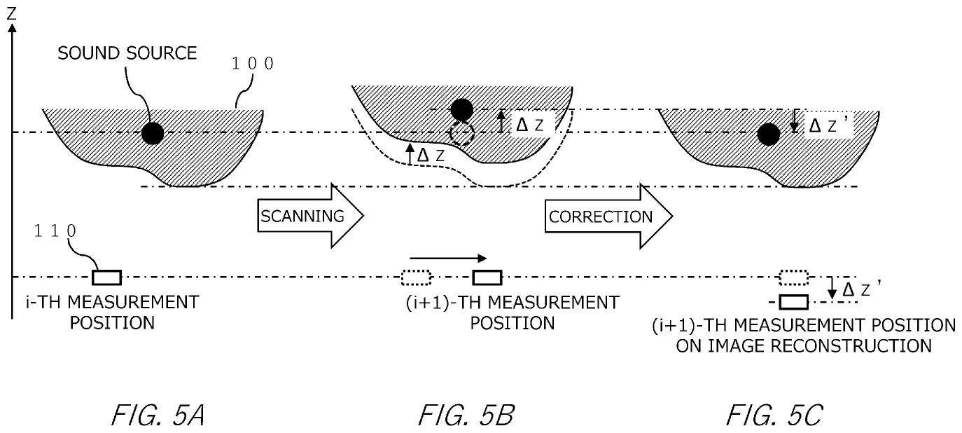

With reference to FIGS. 5A to 5C, the description of equation (2) will be given below. In FIGS. 5A to 5C, the vertical axis represents the position in the z direction. The upper side of the horizontal axis represents the state of the object. For simplification, the object is displaced in the z direction, but the displacement in the xy direction is not assumed. The lower side of the horizontal axis represents how the relative position of the probe to the object is changed during the scanning.

FIGS. 5A to 5B shows a case where the object 100 causes body movement and the displacement with the body movement amount Δz occurs during the second acoustic wave measurement. During this time, the probe is moved from the i-th measurement position to the (i+1)-th measurement position. In the present embodiment, as shown in FIG. 5B, the displacement amount of the sound source (light absorbing materials) inside the object is the same as the body movement amount Δz obtained in S190. When performing the image reconstruction in this situation, sound source images derived from the received signals at the i-th and (i+1)-th measurement positions are back-projected to different positions due to the difference in the sound source positions in FIGS. 5A and 5B. As a result, this causes a reduction of the resolution and contrast, as well as the deformation of the sound source image.

FIG. 5C shows the state of correcting such image degradation. The processing unit 140 shifts the position the probe 110 on the image reconstruction by the body movement correction amount Δz′ to the opposite direction from the body movement amount. Thus, as shown in FIG. 5C, the apparent sound source positions of the i-th and (i+1)-th measurement positions do match. As a result, the body movement is corrected. This process provides the benefit of being able to obtain the object information whose displacement of the object is corrected.

Examples of Display

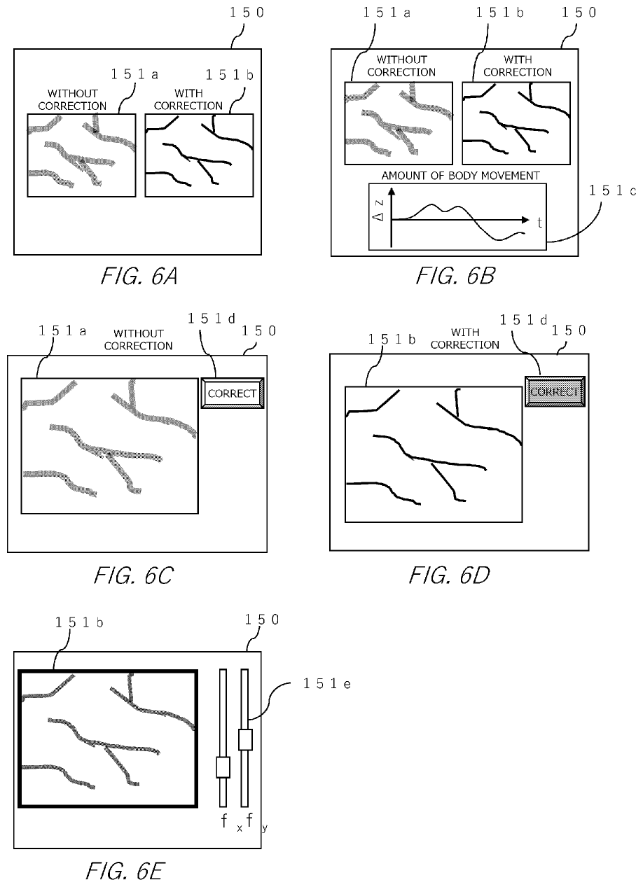

After S200, the display unit 150 may display the initial sound pressure distribution p₀(r) where the body movement is corrected by formula (2). Examples of the display are shown in FIGS. 6A to 6E. FIG. 6A is an example of displaying a window 151a with no body movement correction and a window 151b with the body movement correction. While the decrease in the contrast is observed in the images before the correction, an image with higher contrast less influenced by the body movement is displayed after the correction. An operator can check the effect and accuracy of the correction process by comparing both windows displayed in parallel.

In FIG. 6B, the effect and accuracy can be easily confirmed by displaying the detected body movement amount in a window 151c. Here, the horizontal axis represents the time and the vertical axis represents the body movement amount, but the horizontal axis may also be the measurement position or the scanning distance. FIGS. 6C and 6D is an example of having a button 151d to enable switching the images with or without correction. An operator can switch the display illustrating the state before the correction as shown in FIG. 6C and after the correction as shown in FIG. 6D by clicking the button displayed as GUI. In this case, it becomes easier to compare the images before and after the correction since the two images appear at the same position on the display. For example, a quicker switching of the display is possible by only clicking the button to switch the images while the correction process is running in the background.

FIG. 6C shows a case where a slide bar 151e is available to enter the parameters for the correction. For example, cutoff frequencies for smoothing on the x and the y directions can be set. Any other parameters needed for the correction may be added. The button and the slide bars for switching the display can be adjusted by using a UI of the computer as an input means by an operator.

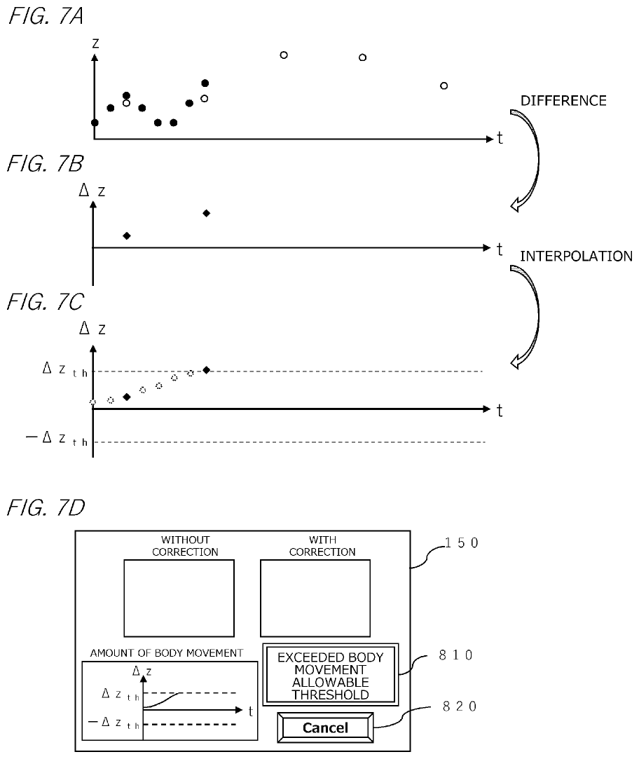

Also, a warning may be displayed on the display unit 150 when a large body movement occurs during the measurement. With reference to FIGS. 7A to 7D, a case will be described where a body movement exceeding an allowable threshold occurs during the second acoustic wave measurement after completing the first acoustic wave measurement.

FIGS. 7A to 7C corresponds to FIGS. 4A to 4C during the second acoustic wave measurement, respectively. In the example of FIGS. 7A to 7D, while calculating the body movement amount Δz at the same timing as the measurement, whether a predetermined allowable threshold Δzₜₕ of the body movement amount exceeds during the second acoustic wave measurement is determined. The scanning is continued if the body movement amount is equal to or less than the allowable threshold, whereas, as shown in FIG. 7D, a warning 810 is displayed on the display unit 150 if the body movement amount exceeds the allowable threshold. In this case, it is preferable that a cancellation button 820 is provided so that an operator can suspend the measurement. Also, if the body movement exceeds the allowable threshold, the measurement may also be aborted on the device side. The allowable threshold can be set by deriving the correction limit of the body movement correction process or the like from a simulation.

Examples of Scanning Paths and Measurement Positions

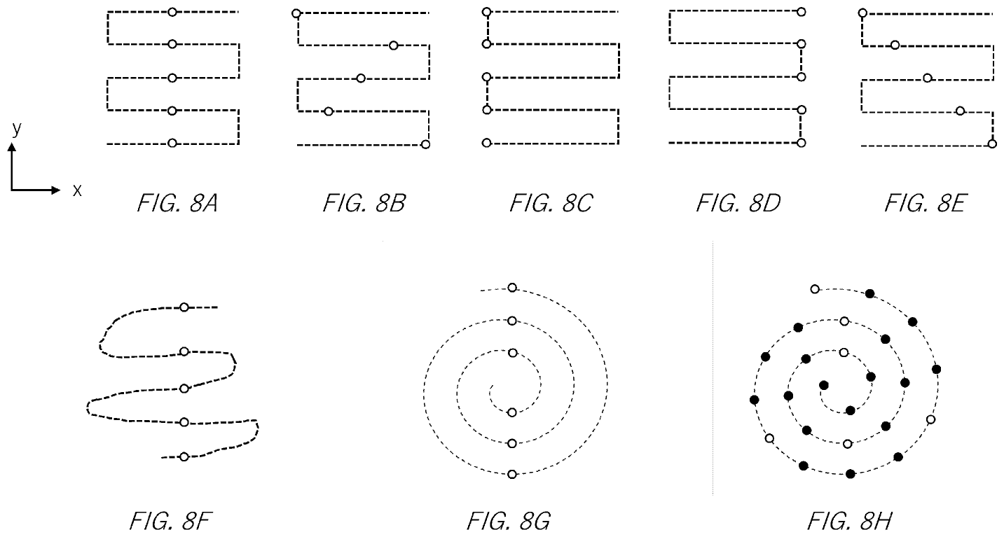

FIGS. 8A to 8H show examples of various embodiments in the first and the second acoustic wave measurement. FIGS. 8A to 8E represent the measurement positions in the first acoustic wave measurement when the same scanning paths and measurement positions as FIG. 3B are employed in the second acoustic wave measurement. Note that, not limited to raster scanning, any scanning method may be used as long as it includes sub-scanning to the sub-scanning direction which intersects the main scanning direction. In FIG. 8A, a group of the measurement positions extracted from every four measurement positions in the second acoustic wave measurement is used as the measurement positions in the first acoustic wave measurement. The above case is preferable because the first scan can be on a straight line. In FIG. 8B, a group of the measurement positions extracted from every five measurement positions in the second acoustic wave measurement is used as the measurement positions in the first acoustic wave measurement.

In the examples of FIGS. 8A and 8B, the measurement positions of the first acoustic wave measurement are arranged equally-spaced with respect to the number of measurement positions of the second acoustic wave measurement. Further, assuming that the measurement positions of the second acoustic wave measurement are arranged evenly with respect to the scanning distance, the measurement positions of the first acoustic wave measurement is also arranged evenly with respect to the scanning distance. Similarly, assuming that the measurement positions of the second acoustic wave measurement are arranged evenly with respect to the scanning time, the measurement positions of the first acoustic wave measurement is also arranged evenly with respect to the scanning time. However, when employing an arrangement evenly spaced with respect to time, it is preferable to consider a case where the scanning speed may not be constant for such switching of the main and sub-scanning occurs in the raster scanning.

FIGS. 8C, 8D, and 8E are examples of the measurement positions in the first acoustic wave measurement arranged on a straight line. The above cases are preferable because the path of the first scan can be set on a straight line.

FIG. 8F is an example in which the measurement positions in the first acoustic wave measurement are defined as intersections between the straight line and the curve when the scanning path of the second scan is any curve.

FIGS. 8G and 8H are examples where the second scan is on a spiral trajectory. In FIG. 8G, the measurement positions of the first acoustic wave measurement are arranged at the intersections between the spiral trajectory and the straight line. In FIG. 8H, a group of the measurement positions extracted from every three measurement positions on the spiral trajectory in the second acoustic wave measurement (black circles) is used as the measurement positions in the first acoustic wave measurement (white circles). Note that in FIG. 8H, a position where a black and a white circle overlap is shown as a white circle.

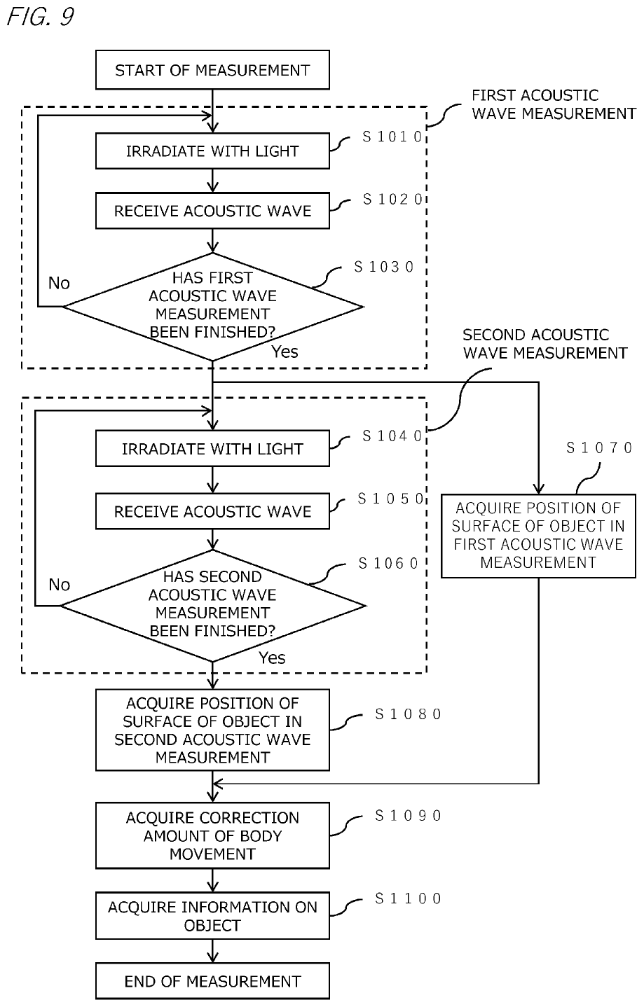

In any case of FIG. 9, the measurement positions in the first and the second acoustic wave measurement are preferred to be arranged in the same position. The accuracy to acquire the body movement amount in FIG. 4B is improved by matching the measurement positions between the first and the second acoustic wave measurements. Here, the match of the position referred to herein may be at any degree of the extent that can be considered to be consistent in relation to the desired accuracy in calculating the body movement amount or the object information.

If the measurement positions between the first and the second acoustic wave measurement do not match, it is preferable to perform a spatial interpolation processing such as corresponding the two nearest measurement positions. In the interpolation of the body movement amount in S190, spline interpolation or polynomial interpolation may be used. Furthermore, as shown in FIGS. 8A, 8B, and 8H, when the measurement positions are evenly spaced with respect to the scanning time (or scanning distance), a high-precision interpolation processing can be used such as sinc interpolation and Lanczos interpolation based on the sampling theorem. Further, even if the measurement positions are not evenly spaced, it is possible to carry out high-precision interpolation based on the unequal-interval sampling theorem. In order to improve the accuracy of the interpolation, it is preferable that the measurement positions in the second acoustic wave measurement where the distance between the measurement positions is shorter than half of the period of the body movement are used as the measurement positions in the first acoustic wave measurement.

The correction method of the long-period body movement according to the present invention may be combined with the correction method of the short-period body movement described in Non-Patent Document 1. In Non-Patent Document 1, the body movement amount of a short period is obtained by smoothing the extracted object surface shape and calculating the difference between the original object surface shape and the smoothed shape. When combining this method with the body movement correction of a long period of the present invention, it is preferable that the filtering characteristics used for smoothing are exclusive. The body movement amount correction of a short period in Non-Patent Document 1 is represented by formula (3), where Δzₛₘₐₗₗ is the body movement amount of a short period, z(x, y) is z coordinates on the object surface, z′(x, y) is z coordinates on the smoothed object surface, h(x, y) is a smoothing filter, and the capital letters represent a frequency domain. If the maximum value of H is standardized to 1, formula (3) indicates that the components passing through the smoothing filter become the body movement amount of a short period.

$$\begin{matrix}Δ {z}_{small} ( x , y ) = z ( x , y ) - {z}^{′} ( x , y ) \\ = z ( x , y ) - z ( x , y ) * h ( x , y ) \\ = z ( x , y ) - {ℱ}_{2}^{- 1} ( Z ( {f}_{x} , {f}_{y} ) H ( {f}_{x} , {f}_{y} ) ) \\ = {ℱ}_{2}^{- 1} ( Z ( {f}_{x} , {f}_{y} ) ) - {ℱ}_{2}^{- 1} ( Z ( {f}_{x} , {f}_{y} ) H ( {f}_{x} , {f}_{y} ) ) \\ = {ℱ}_{2}^{- 1} ( Z ( {f}_{x} , {f}_{y} ) ( 1 - H ( {f}_{x} , {f}_{y} ) ) )\end{matrix} \\ {ℱ}_{2}^{- 1} \text{:} Two \text{-} dimensional inverse Fourier transform \\ * \text{:} Convolution \qquad ( 3 )$$

Therefore, in the body movement correction of a long period, the processing unit 140 can correct the short-period body movement and exclusive components by performing the processing in S190 and thereafter on z′ (x, y) obtained by applying a filter h(x, y) to z(x, y) extracted in S170 and S180. For example, in FIG. 3A, the smoothing may only apply on h_{y}(y), which represents h(x, y) in the y direction. Thus, the body movement correction with high precision can be performed by preventing repeated corrections by the long-period and the short-period body movement corrections.

Therefore, according to the present embodiment, the body movement can be corrected by obtaining the object surface shape by the first acoustic wave measurement within a short time period in order to generate the object information with high precision.

### Second Embodiment

In the present embodiment, the body movement correction amount is obtained by spatially interpolating the object surface shape. Note that the same reference numerals will designate the same components as those of the first embodiment without further explanation.

Configuration of Present Object Information Acquisition Apparatus

A schematic diagram of the object information acquisition apparatus and the surrounding configuration of the processing unit according to the present embodiment are the same as those of the first embodiment described above. In the present embodiment, the contents of the processing to be executed by the processing unit 140 in FIG. 1A are different.

Object Information Acquisition Method

Next, with reference to FIG. 9, each step of the object information acquisition method according to the present embodiment will be described. Each step is executed by controlling the operation of each configuration of the object information acquisition apparatus by the processing unit 140. S1010, S1020, S1040 to S1080, and S1100 are the same as S110, S120, S140 to S180, and S200 in the first embodiment, respectively, and need not be repeated.

Step S1030: Step for Determining Completion of First Acoustic Wave Measurement

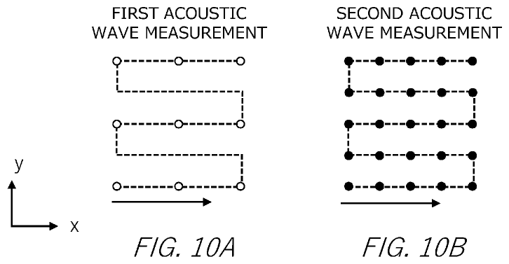

The object information acquisition apparatus scans the measurement positions with the probe 110 and/or the irradiation unit 120 and repeats the processing of steps S1010 and S1020 until the measurement completes at all the measurement positions in FIG. 10A. Then, in step S1030, the completion of the first acoustic wave measurement is to be determined.

The measurement position in the first acoustic wave measurement of the present embodiment is shown as white circles in FIG. 10A. The measurement position in the second acoustic wave measurement of the present embodiment is shown as black circles in FIG. 10B. In order to spatially interpolate the object surface position obtained in the first acoustic wave measurement, it is desirable that a group of the measurement positions in the first acoustic wave measurement shown in FIG. 10A includes the scanning region in the second acoustic wave measurement (a region surrounded by black circles shown in FIG. 10B). For example, if the first scan and the second scan draw the same path, it is preferable that the starting and the ending measurement positions of the first acoustic wave measurement respectively overlap the starting and the ending measurement positions of the second acoustic wave measurement. In that case, the measurement region in the first acoustic wave measurement and the measurement region in the second acoustic wave measurement can be considered as mutually inclusive.

Even if it is not mutually inclusive, it is preferable that the range in which the measurement area surrounded by the measurement position group in the first acoustic wave measurement and the measurement area surrounded by the measurement position group in the second acoustic wave measurement overlap as broad as possible.

Similar to the first embodiment, the number of measurement positions in the first acoustic wave measurement is fewer than the number of measurement positions in the second acoustic wave measurement. Therefore, since the first acoustic wave measurement is completed in a shorter period of time than the second acoustic wave measurement, it becomes possible to obtain the object surface information (object shape information) with less influenced by the long-period body movements based on the received signals obtained in the first acoustic wave measurement.

Step S1090: Step for Acquiring Body Movement Correction Amount

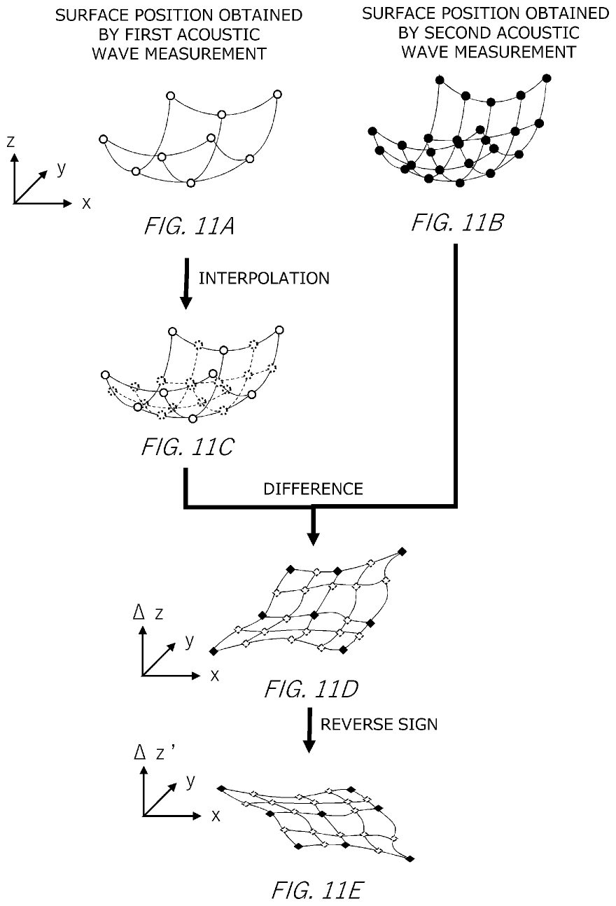

In this step, a body movement correction amount is acquired by using the object surface position obtained in S1070 and S1080. FIG. 11A shows the object surface position obtained in the first acoustic wave measurement. The white circles correspond to each measurement position in FIG. 10A. FIG. 11B shows the object surface position obtained in the second acoustic wave measurement. The black circles correspond to each measurement position in FIG. 10B. As can be seen by comparing the figures, the number of the point indicated by the white circle is fewer than those indicated by the black circle.

Therefore, the processing unit 140 performs interpolation of the missing measurement position using the data at each measurement position in FIG. 11A. As a result, as shown in FIG. 11C, the first surface shape information with the interpolated object surface position is obtained. Subsequently, the processing unit 140 calculates a difference by subtracting the first surface shape information in FIG. 11C from the second surface shape information in FIG. 11B. As a result, the body movement amount Δz shown in FIG. 11D is obtained. The body movement of the present embodiment is acquired as body movement distribution information. Subsequently, the processing unit 140 obtains the body movement correction amount Δz′ shown in FIG. 11E by reversing the sign in FIG. 11D.

Therefore, according to the present embodiment, it is possible to obtain information needed to correct the body movement based on the object surface shape obtained in the first and the second acoustic wave measurement. Since the body movement correction amount of the present embodiment is acquired as distribution information along the shape of the object, it is possible to improve the accuracy of the correction to generate the object information with high precision.

### Third Embodiment

In the present embodiment, the body motion correction amount is obtained by performing the high-speed first acoustic wave measurement utilizing the relation between the repetition frequency (pulse repetition rate, PRR) of the light sauce and the energy of the emitting light pulse (pulse energy, PE). Note that the same reference numerals will designate the same components as those of the first embodiment and the second embodiment without further explanation.

Configuration of Present Object Information Acquisition Apparatus

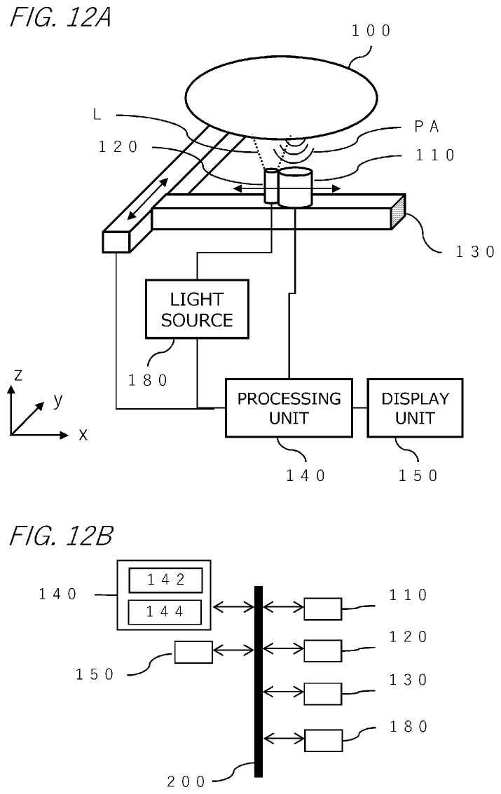

FIG. 12A is a schematic diagram of the object information acquisition apparatus according to the present embodiment. FIG. 12B is a schematic diagram illustrating the relation between the processing unit 140 and the surrounding configuration. In the present embodiment, a portion of the processing contents performed by the processing unit 140 is different from those in the first embodiment. Since the present embodiment has a feature to control a light sauce, a light source 180 is clearly indicated in these figures. The light source 180 emits light from the irradiation unit 120 in accordance with the control of the processing unit 140. However, as long as it can perform the light control required for the present embodiment, light guided from an external light source may be used in the configuration.

Light Source 180

In addition to the features of the light source described in the first embodiment, the light source 180 can adjust the PRR. In the present embodiment, the PRR is to be adjusted in the first and the second acoustic wave measurement.

Object Information Acquisition Method

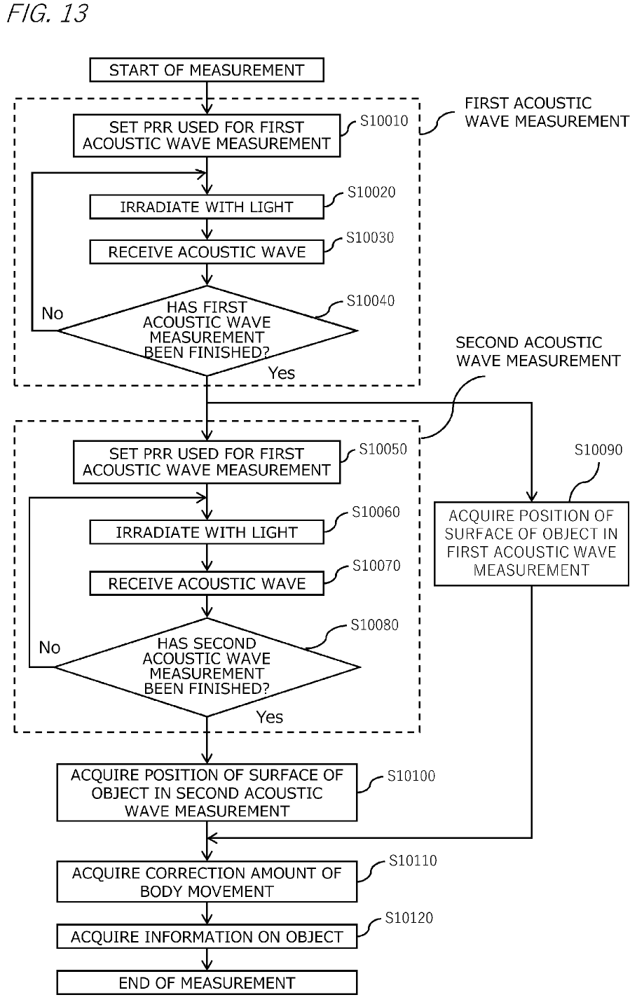

Next, with reference to FIG. 13, each step of the object information acquisition method according to the present embodiment will be described. It should be noted that each step is executed by controlling the operation of each configuration of the object information acquisition apparatus by the processing unit 140. S10020, S10030, and S10060 to S10120 are the same as S110, S120, and S140 to S200 in the first embodiment, respectively, and need not be repeated.

S10010: Step for Setting PRR of Light Source to Value Used in First Acoustic Wave Measurement

In this step, the PRR of the light source is set to a value greater than the value set in S10050 (the value used in the scanning of the second acoustic wave measurement). That is, the repetition frequency of the first acoustic wave measurement is higher than those of the second acoustic wave measurement. As a result, the pulsed light irradiation in S10020 and the acoustic wave reception in S10030 are performed at a relatively short period.

Step S10040: Step for Determining Completion of First Acoustic Wave Measurement

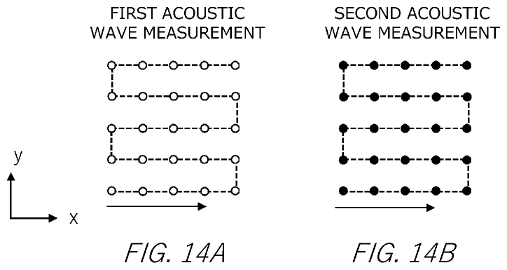

In this step, the completion of the first acoustic wave measurement is to be determined. The steps S10020 and S10030 are repeated by scanning the measurement positions with the probe 110 and/or the irradiation unit 120 until the measurements are completed at all the measurement positions (white circles in FIG. 14A) where the received signals should be obtained in the first acoustic wave measurement. In the present embodiment, the PRR of the first acoustic wave measurement is greater than those of the second acoustic wave measurement. Therefore, the first acoustic wave measurement can be completed in a shorter period of time than the second acoustic wave measurement even if the measurement position groups in the first and the second acoustic wave measurement are the same, as FIGS. 14A and 14B. Therefore, it is possible to obtain the received signals with less influenced by the long-period body movements in the first acoustic wave measurement. Furthermore, since the number of the measurement positions can be increased than those in the first or the second embodiment, it is possible to improve the accuracy of the body movement correction amount. Note that the number of measurement positions does not necessarily have to be the same between the first and the second acoustic wave measurement. Similar to the above embodiments, the time for the first acoustic wave measurement can be shortened by reducing the number of measurement positions in the first acoustic wave measurement.

S10050: Step for Setting PRR of Light Source to Value Used in Second Acoustic Wave Measurement

In this step, the PRR of the light source is set to a value smaller than the value set in S10010 (the value used in the scanning of the first acoustic wave measurement). That is, the repetition frequency of the second acoustic wave measurement is lower than those of the first acoustic wave measurement. As a result, the pulsed light irradiation in S10060 and the acoustic wave reception in S10070 are performed at a relatively long period.

Here, the features of the settings of the PRR and PE in the present embodiment will be described. FIG. 15A is a schematic view of the object in cross-section. The object surface is composed of stratum corneum in the epidermis or the like, a melanin layer containing melanin pigments presents underneath (in deep). In addition, there is dermis and subcutaneous tissue with a layer containing blood vessels in a deeper portion of the skin. FIG. 15B is a schematic view illustrating the amount of light reached to the inside of the object in the first acoustic wave measurement. FIG. 15C is a schematic view illustrating the amount of light reached to the inside of the object in the second acoustic wave measurement. In FIGS. 15B and 15C, the darker hatching indicates the greater amount of light.

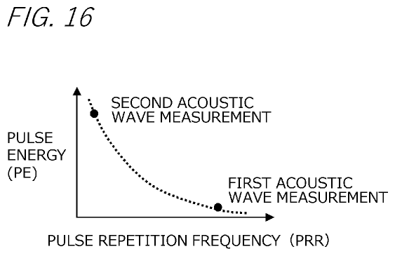

Here, in the first acoustic wave measurement, it is only necessary to obtain the shape information on the object surface. Therefore, in FIG. 15B, the set value of PE is small and just enough for the light to be reached to the melanin layer right below the object surface. On the other hand, in the second acoustic wave measurement, it is necessary to reach the light to the layer including blood vessels (object's deep) which is often the target for diagnosis. Therefore, in FIG. 15C, the setting value of the PE is greater than those in FIG. 15B. Thus, by setting smaller PE in the first acoustic wave measurement than those in the second acoustic wave measurement, it becomes possible to satisfy the standards (such as MPE) determined for the safety of the object, even if the set value of PRR in the first acoustic wave measurement is increased. Further, as shown in FIG. 16, the light source 180 generally has PRR and PE that tends to be reduced monotonically. Therefore, since the first acoustic wave measurement can set the PRR higher than the second acoustic wave measurement, it is possible to shorten the time required for the scanning.

When calculating the body movement correction amount in step S10110 of the present embodiment, interpolation may be performed either as temporal or distance-based interpolation described in the first embodiment or as spatial interpolation described in the second embodiment.

Thus, according to the present embodiment, the object surface shape information with high precision can be obtained by setting the light source PRR in accordance with the contents of the first acoustic wave measurement. As a result, since the accuracy of the calculation for the body movement correction information is also improved, it is possible to generate the object information with high precision.

### Fourth Embodiment

### Other Configuration Examples of Object Information Acquisition Apparatus

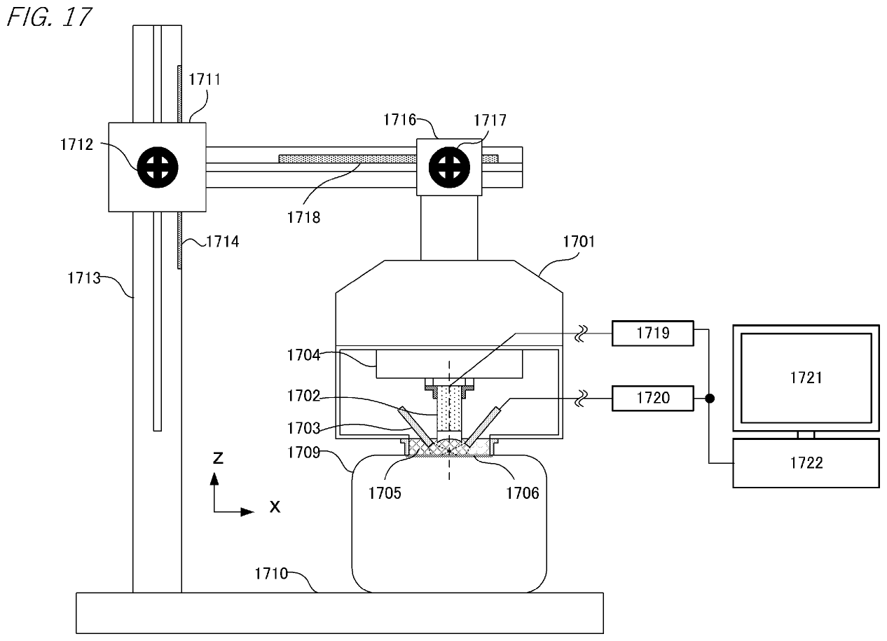

FIG. 17 is a schematic diagram of another configuration example of the object information acquisition apparatus to which the present invention is applicable. The object information acquisition apparatus in FIG. 17 contains a probe unit 1701, a probe unit holding mechanism 1713, a signal acquisition unit 1719, a light source 1720, a device control unit 1722, and a display device 1721.

The probe unit 1701 is a unit for irradiating the object with light, and for receiving acoustic waves generated from the object. The probe unit 1701 contains a light irradiation unit 1703 for irradiating the object with light, an acoustic probe 1702 for receiving acoustic waves, and a scanning mechanism 1704. The light irradiation unit 1703 and the acoustic probe 1702 is configured integrally movable by the scanning mechanism 1704. The probe unit 1701 and the object 1709 is in contact through a biological contact film 1706. In the following description, the biological contact film 1706 is referred to as “contacting surface (between the probe unit and the object)”. The light irradiation unit corresponds to the irradiation means in the present embodiment. The scanning mechanism corresponds to the scanning unit in the present embodiment.

The biological contact film 1706 is a film composed of polyethylene terephthalate or the like. The biological contact film 1706 is preferably a material having a strength not deformed by the object, as well as having a property of transmitting light and acoustic waves. In the present embodiment, the opening of the biological contact film is 30 mm×30 mm. Between the biological contact film 1706 and the acoustic probe 1702, water 1705 as an acoustic matching agent (acoustic propagation medium) is stored. Note that the preferred thickness of the biological contact film 1706 is about 100 microns in order to avoid multiple reflections of the acoustic wave within the film.

The probe unit holding mechanism 1713 is a mechanism for holding and moving the probe unit 1701. The probe unit holding mechanism 1713 includes a Z-axis stage 1711 for the movement on the Z axis, an X-axis stage 1716 for the movement on the X axis. The Z-axis stage 1711 is configured to be movable by a Z-axis handle 1712. Therefore, the probe unit 1701 can be moved on the Z axis with respect to the object 1709. The position of the Z-axis stage is detected by a Z-axis encoder 1714. Therefore, it is possible to calculate the position of the probe unit on the Z axis. Similarly, the X-axis stage 1716 is movable by an X-axis handle 1717. Therefore, the probe unit 1701 can be moved on the X axis with respect to the object 1709. The position of the X-axis stage is detected by an X-axis encoder 1718. Therefore, it is possible to calculate the position of the probe unit on the X axis.

The light source 1720 is a device for generating pulsed lights to irradiate the object. As the light source 1720, the same light source device as in the above embodiment can be used. The wavelength, pulse length or the like may also be set in the same manner as in the above embodiment. It should be noted that the timing of the light irradiation, waveform, intensity, or the like is controlled by the device control unit 1722 described below. In this configuration example, the pulse width is set to 10 nanoseconds and the repetition frequency is set to 200 Hz. Further, a YAG laser that can switch the wavelength of 532 nm and 1064 nm is used. Although 532 nm is a wavelength absorbed well in the living body, the photoacoustic device in the present embodiment can use the aforementioned wavelength since the photoacoustic device only measures the position from the object surface to 5 mm below. Note that by using the wavelength of 1064 nm, it is also possible to identify blood vessels and melanin.

The light emitted from the light source 1720 irradiates the object 1709 using an optical fiber of the light irradiation unit 1703. It should be noted that the optical fiber may be arranged in a ring shape around the acoustic probe 1702. Further, a light that can be spread to a certain area is preferred over a light that can be gathered by a lens in terms of the safety to the living body or of being able to expand the diagnostic area.

The acoustic probe 1702 is a means to receive the acoustic waves coming from the inside of the object and to convert it to the electrical signals in time-series. As the acoustic probe 1702, the same probes or transducers as in the above embodiment can be used. The wavelength, receiving system or the like may also be set in the same manner as in the above embodiment.

The acoustic probe 1702 in the present embodiment is a probe in an acoustic focus type consisting of PZT and an acoustic lens and is able to efficiently receive acoustic waves generated from a predetermined focus. The diameter is 6 mm and the center frequency is 50 MHz. An acoustic lens made from quartz glass is assembled to the tip of the probe and its numerical aperture is 0.6. Resolution on the XY plane is determined by the performance of the acoustic probe 1702 and is about 60 μm in the present embodiment. The resolution in the depth direction is about 80 percent of the wavelength that can be detected (about 30 μm). The focus is in a position 4 mm away from the probe and is consistent with the position of the biological contact film 1706. Note that in some cases, the position of the focus may be better to be placed closer to the probe side, for example, by 0.5 mm.

The signal acquisition unit 1719 is a means for amplifying the analog electrical signals obtained by the acoustic probe 1702 and converting it to the digital electrical signals. The signal acquisition unit 1719 may be configured using an amplifier for amplifying the received signals and an A/D converter for digitally converting the analog signals. The signal acquisition unit 1719 may also be composed of a plurality of processors and arithmetic circuits.

In the present embodiment, the sampling frequency is 500 MHz and the number of sampling is 8192. The sampling is started after a predetermined time passed from the generation of a trigger signal representing the timing of the light irradiation. It should be noted that the signal acquisition unit 1719 may further have a memory such as FIFO for storing the received signals and an arithmetic circuit such as FPGA chip. The device control unit 1722 may be realized by a general-purpose computer or a workstation designed exclusively.

The device control unit 1722 is a means to obtain the object information (means to generate images in the present invention) such as the light absorption coefficient and oxygen saturation inside the object by performing the reconstruction process based on the digitally converted signals (photoacoustic signals). Specifically, the device control unit 1722 generates an initial sound pressure distribution in the three-dimensional object from the collected electrical signals.

Further, the device control unit 1722 also generates a three-dimensional light intensity distribution inside the object based on the information on the amount of light for irradiating the object. The three-dimensional light intensity distribution can be derived by solving the light diffusion equation from the information on the two-dimensional light intensity distribution. Further, the absorption coefficient distribution inside the object can be obtained by using the initial sound pressure distribution in the object generated from the photoacoustic signals and the three-dimensional light intensity distribution. Further, the oxygen saturation distribution in the object can be derived by calculation from the absorption coefficient distribution at a plurality of wavelengths. The processing unit corresponds to the control unit and information acquiring unit in the present embodiment.

It should be noted that the device control unit 1722 may have functions to carry out the desired process such as the calculation of the light intensity distribution, the information processing necessary to obtain the optical coefficient on the background, or the signal correction. Further, the device control unit 1722 may obtain instructions on changes in the measurement parameters, start and end of the measurement, the selection of the image processing method, storing patient information or images, or the data analysis via a display device or an input interface described below. The device control unit 1722 is also a means for controlling each component of the photoacoustic device. For example, the device control unit 1722 commands the control of the entire device such as, for example, the irradiation of the object with light, the reception of the acoustic waves and photoacoustic signals, the movement of the probe unit.

The device control unit 1722 may be composed of a computer having a CPU and RAM, a nonvolatile memory, and a control port. The control is performed by the CPU executing the program stored in the nonvolatile memory. The device control unit 1722 may be realized by a general-purpose computer or a workstation designed exclusively. The unit responsible for the arithmetic function of the device control unit 122 may be composed of a processor such as a CPU or GPU, an arithmetic circuit such as an FPGA chip. These units are not only composed of a single processor or an arithmetic circuit but may be composed of a plurality of processors or arithmetic circuits.

The unit responsible for the storage function of the device control unit 1722 may be a volatile medium such as a ROM, a non-transitory storage medium such as a magnetic disk or flash memory, or a volatile medium such as RAM. Note that the storage medium in which the program is stored is a non-transitory storage medium. It should be noted that these units are not only composed of one storage medium but may be composed of a plurality of storage medium. The unit responsible for the control function of the device control unit 1722 is composed of an arithmetic element such as a CPU.

The display device 1721 is a means for displaying the acquired and processed information by the device control unit 1722, typically a display device. The display device 1721 may be a plurality of devices, a plurality of display units in a single device, or a device capable of a parallel display. It should be noted that it is preferable that the display device 1721 uses a display with 30-inch or more in size capable of color displaying at high resolution and the contrast ratio is greater than 1000:1. The display device corresponds to the display means in the present embodiment.

As described above, the object information acquisition apparatus in the present configuration example is arranged inside the probe unit apart from the probe unit holding mechanism holding and moving the probe unit and equips a scanning mechanism to move the acoustic probe and the light irradiation unit. In such a configuration, it is possible to perform a relatively short-period measurement as the first acoustic wave measurement and a relatively long-period measurement as the second acoustic wave measurement by scanning the acoustic probe and the light irradiation unit based on the movement control of the scanning mechanism. As a result, high-precision photoacoustic image data can be obtained with less influence of the displacement of the object such as body movement by correcting the object information obtained in the second acoustic wave measurement.

### Fifth Embodiment

The present invention is applicable not limiting to the photoacoustic imaging apparatus but also to any apparatus capable of obtaining the object surface position. For example, in the case of an ultrasonic echo imaging apparatus or ultrasonic tomography apparatus, the object surface position can be obtained from the acoustic impedance difference between the object surface and the acoustic matching agent.

Here, an ultrasonic echo imaging apparatus will be described as an example. The ultrasonic echo imaging apparatus performs a relatively short-period measurement as the first ultrasonic echo measurement and performs a relatively long-period measurement as the second ultrasonic echo measurement. Then, the body movement correction information is calculated based on the result of the first ultrasonic echo measurement to correct the acoustic impedance distribution obtained in the second ultrasonic echo measurement. At this time, a method such as reducing the number of measurement positions or increasing the frequency of the echo transmission can be used to shorten the time for the first ultrasonic echo measurement.

Further, while the correction on the z direction in FIGS. 1A and 1B has been described in each of the above embodiments, it may be the corrections on the x or y directions. For example, in the probe capable of forming a focusing point of the acoustic waves, the body movement amount on the x and y directions can be obtained by matching the reception signals corresponding to the position of the focusing point at the measurement position in the first acoustic wave measurement and those at the corresponding second acoustic wave measurement. As a probe capable of forming a focusing point of the acoustic waves, for example, there is an array transducer, a focusing transducer or the like.

The present invention has been described above in detail with reference to the specific embodiments. However, the present invention is not limited to the above specific embodiments and the embodiment can be modified without departing from the technical concept of the present invention. As described above, according to the present invention, it is possible to provide an image with less calculation time while improving the image quality of photoacoustic imaging and ultrasonic echo imaging.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2018-171759, filed on Sep. 13, 2018, which is hereby incorporated by reference herein in its entirety.

What is claimed is:

1. An object information acquisition apparatus, comprising:
a probe configured to receive an acoustic wave generated from an object and to output a signal;
a scanning unit configured to scan the object with the probe by changing a position of the probe relative to the object;
a control unit configured to control execution of acoustic wave measurement on the object; and
an information acquiring unit configured to acquire characteristic information on the object by using the signal, wherein
the control unit executes first acoustic wave measurement and second acoustic wave measurement for a predetermined region of the object, and the first acoustic wave measurement is executed within a shorter period of time than the second acoustic wave measurement, and
the information acquiring unit acquires displacement information indicating displacement of the object by using a first signal acquired by the first acoustic wave measurement and a second signal acquired by the second acoustic wave measurement.

2. The object information acquisition apparatus according to claim 1, wherein the information acquiring unit acquires first object shape information by using the first signal, acquires second object shape information by using the second signal, and acquires the displacement information by using the first object shape information and the second object shape information.

3. The object information acquisition apparatus according to claim 2, wherein the information acquiring unit acquires the characteristic information by using the second signal and corrects the characteristic information by using the displacement information.

4. The object information acquisition apparatus according to claim 1, wherein the control unit performs control so that a number of measurement positions in the first acoustic wave measurement is smaller than a number of measurement positions in the second acoustic wave measurement.

5. The object information acquisition apparatus according to claim 4, wherein the control unit performs control so that a plurality of measurement positions in the first acoustic wave measurement each overlap any one of a plurality of measurement positions in the second acoustic wave measurement.

6. The object information acquisition apparatus according to claim 4, wherein the control unit performs control so that a region surrounded by a plurality of measurement positions in the first acoustic wave measurement includes a region surrounded by a plurality of measurement positions in the second acoustic wave measurement.

7. The object information acquisition apparatus according to claim 1, wherein the scanning unit is configured to:
move the probe on a raster trajectory or a trajectory including a curve during the second acoustic wave measurement; and
move the probe in a direction intersecting the raster trajectory or the trajectory including a curve during the first acoustic wave measurement.

8. The object information acquisition apparatus according to claim 2, wherein the information acquiring unit acquires a position of a surface of the object at a plurality of measurement positions in the first acoustic wave measurement, acquires a position of the surface of the object at a plurality of measurement positions in the second acoustic wave measurement, and acquires the displacement information on the basis of the positions of the surface acquired in the first acoustic wave measurement and the positions of the surface acquired in the second acoustic wave measurement.

9. The object information acquisition apparatus according to claim 1, wherein the information acquiring unit acquires the displacement information by interpolation processing on missing measurement positions in the first acoustic wave measurement in a case where a number of measurement positions in the first acoustic wave measurement is smaller than a number of measurement positions in the second acoustic wave measurement.

10. The object information acquisition apparatus according to claim 1, further comprising an irradiation unit configured to irradiate the object with pulsed light to generate the acoustic wave.

11. The object information acquisition apparatus according to claim 10, wherein a repetition frequency of the pulsed light in the first acoustic wave measurement is higher than a repetition frequency of the pulsed light in the second acoustic wave measurement.

12. The object information acquisition apparatus according to claim 10, wherein energy of the pulsed light in the first acoustic wave measurement is lower than energy of the pulsed light in the second acoustic wave measurement.

13. The object information acquisition apparatus according to claim 1, wherein the acoustic wave is a wave transmitted from the probe toward the object and reflected on the object.

14. The object information acquisition apparatus according to claim 3, further comprising a display unit configured to display an image based on the characteristic information,
wherein the display unit displays an uncorrected image and a corrected image side by side or in a switchable manner.

15. A method of controlling an object information acquisition apparatus, comprising:
an output step of receiving, by a probe, an acoustic wave generated from an object and outputting a signal;
a scanning step of scanning the object with the probe by changing a position of the probe relative to the object;
a control step of controlling execution of acoustic wave measurement on the object; and
an information acquisition step of acquiring characteristic information on the object by using the signal, wherein
the control step includes executing first acoustic wave measurement and second acoustic wave measurement for a predetermined region of the object, and the first acoustic wave measurement is executed within a shorter period of time than the second acoustic wave measurement, and
the information acquisition step includes acquiring displacement information indicating displacement of the object by using a first signal acquired by the first acoustic wave measurement and a second signal acquired by the second acoustic wave measurement.

16. The method of controlling an object information acquisition apparatus according to claim 15, wherein the information acquisition step includes acquiring first object shape information by using the first signal, acquiring second object shape information by using the second signal, and acquiring the displacement information by using the first object shape information and the second object shape information.

17. The method of controlling an object information acquisition apparatus according to claim 16, wherein the information acquisition step includes acquiring the characteristic information by using the second signal and correcting the characteristic information by using the displacement information.

18. The method of controlling an object information acquisition apparatus according to claim 15, wherein the control step includes performing control so that a number of measurement positions in the first acoustic wave measurement is smaller than a number of measurement positions in the second acoustic wave measurement.

19. The method of controlling an object information acquisition apparatus according to claim 18, wherein the control step includes performing control so that a plurality of measurement positions in the first acoustic wave measurement each overlap any one of a plurality of measurement positions in the second acoustic wave measurement.

20. The method of controlling an object information acquisition apparatus according to claim 18, wherein the control step includes performing control so that a region surrounded by a plurality of measurement positions in the first acoustic wave measurement includes a region surrounded by a plurality of measurement positions in the second acoustic wave measurement.

21. The method of controlling an object information acquisition apparatus according to claim 15, wherein the scanning step includes:
moving the probe on a raster trajectory or a trajectory including a curve during the second acoustic wave measurement; and
moving the probe in a direction intersecting the raster trajectory or the trajectory including a curve during the first acoustic wave measurement.

22. The method of controlling an object information acquisition apparatus according to claim 16, wherein the information acquisition step includes acquiring a position of a surface of the object at a plurality of measurement positions in the first acoustic wave measurement, acquiring a position of the surface of the object at a plurality of measurement positions in the second acoustic wave measurement, and acquiring the displacement information on the basis of the positions of the surface acquired in the first acoustic wave measurement and the positions of the surface acquired in the second acoustic wave measurement.

23. The method of controlling an object information acquisition apparatus according to claim 15, wherein the information acquisition step includes acquiring the displacement information by interpolation processing on missing measurement positions in the first acoustic wave measurement in a case where a number of measurement positions in the first acoustic wave measurement is smaller than a number of measurement positions in the second acoustic wave measurement.

24. The method of controlling an object information acquisition apparatus according to claim 15, wherein
the object information acquisition apparatus further includes an irradiation unit, and
the method further comprises an irradiation step of irradiating, by the irradiation unit, the object with pulsed light to generate the acoustic wave.

25. The method of controlling an object information acquisition apparatus according to claim 24, wherein a repetition frequency of the pulsed light in the first acoustic wave measurement is higher than a repetition frequency of the pulsed light in the second acoustic wave measurement.

26. The method of controlling an object information acquisition apparatus according to claim 24, wherein energy of the pulsed light in the first acoustic wave measurement is lower than energy of the pulsed light in the second acoustic wave measurement.

27. The method of controlling an object information acquisition apparatus according to claim 15, wherein the acoustic wave is a wave transmitted from the probe toward the object and reflected on the object.

28. The method of controlling an object information acquisition apparatus according to claim 17, further comprising a display unit, wherein
the method further comprises a display step of displaying, by the display unit, an image based on the characteristic information, and
the display step includes displaying an uncorrected image and a corrected image side by side or in a switchable manner.
